(19) United States
(12) Patent Application Publication
(10) Pub. No.: US 2005/0107710 A1
(43) Pub. Date: May 19, 2005

(54) Pulse wave analyzing method, pulse wave analyzing software, and so forth

(76) Inventor: Ryu Nakayama, Nishinomiya-shi (JP)

Correspondence Address: OLIFF & BERRIDGE, PLC, P.O. BOX 19928, ALEXANDRIA, VA, 22320, US

(21) Appl. No.: 10/512,310

(22) PCT Filed: Apr. 23, 2003
(86) PCT No.: PCT/JP03/05186

(30) Foreign Application Priority Data
Apr. 24, 2002 (JP) 2002-121657

(51) Int. Cl. A61B 5/02

(52) U.S. Cl. 600/500; 600/504

(57) ABSTRACT

An object of the present invention is to provide a pulse wave analyzing software and a pulse wave analyzing apparatus each of which can extract, from a pulse wave, more information related to an artery.

When a series of formulas developed by the Inventor are applied to pulse wave data, electrocardiogram data, and phonocardiogram data that are measured as time elapses, new parameters related to an artery (e.g., a volume elasticity modulus (Km), a Young's modulus (E_{A}), etc.) can be calculated. These parameters can be used to evaluate functions of a living being in a non-invasive, accurate, easy, and quick fashion.

### TECHNICAL FIELD

The present invention relates to a software etc. that utilizes a pulse wave propagating in an artery of a living being and evaluates information related to the artery.

### BACKGROUND ART

There has conventionally been known (from, e.g., Japanese Patent Application Publication No. 2001-161649 or Japanese Patent Application Publication No. 2001-340306) an apparatus that measures a pulse wave of an artery of an upper limb, a lower limb, etc. of a living being, extracts, from the pulse wave data, information related to an arterial system of the living being, and evaluates a condition of the arterial system (e.g., a degree of arteriostenosis). Since this apparatus is non-invasively used on the living being and takes a considerably short time only to measure the pulse wave, the living being feels little discomfort. Thus, the apparatus enjoys a high usability.

However, the data obtained from the pulse wave by a software employed by the above-indicated apparatus consist mainly of an ankle brachial blood-pressure index (ABI) and a pulse wave velocity (PWV). Thus, it could be said that the information is not sufficiently utilized.

More specifically explained, a pulse wave must include much information related to an artery of a living being. For example, the pulse wave reflects information related to a thickness, an inner radius, an outer radius, a hardness, etc. of the artery. Since, however, an analyzing method implemented by the conventional software is not satisfactorily well schemed, it cannot extract sufficiently much information from the pulse wave. This means that the measured pulse wave data are not sufficiently utilized.

It is therefore an object of the present invention to provide a software that can extract, from a pulse wave, more information related to an artery, and a computer etc. that incorporates the software.

### DISCLOSURE OF THE INVENTION

For many years, the Inventor has continued researches concerning pulse waves, and has been confident that a pulse wave contains much hidden information related to an artery. After the Inventor has performed intensive studies to extract the hidden information, he has succeeded in deriving calculating formulas, described below, and basically completed the present invention:

According to the present invention, there is provided a software that calculates at least one parameter selected from an effective circulating volume (ECV) and a Young's modulus (E), by applying a rheological analysis to pulse wave data measured at two or more different portions of a living being.

The “two or more different portions” may be arbitrarily selected from a carotid artery, a head's artery, a right upper limb, a left upper limb, a right lower limb, and a left lower limb. To obtain much information related to a living being, it is desirable to collect pulse wave data at as many as possible portions of the living being. In the case where a commercially available pulse wave data collecting device is used, pulse wave data obtained from three portions, i.e., a right upper limb, a right lower limb, and a left lower limb, or four portions, i.e., those three limbs and a left upper limb, can be utilized.

The “pulse wave data” mean data obtained by a timewise pulse wave analysis of an arterial system where a volume pulse wave (i.e., a strain) produced by a pressure pulse wave (i.e., a stress) propagated by a heart stroke corresponds, one to one, to the pressure pulse wave, and contain parameters related to time, blood pressure, and volume. The pulse wave data needs to be combined with electrocardiogram data and phonocardiogram data so as to provide a combination of data (i.e., a set of data that assure that the pulse wave data, the electrocardiogram data, and the phonocardiogram data can be compared with each other at each time point on a common time axis). The pulse wave data, the electrocardiogram data, and the phonocardiogram data may be given in the form of digital data recorded on a recording medium such that the data can be read by a computer, or in the form of analog data recorded with a pen recorder on a recording-chart sheet.

The “rheological analysis” is a science related to deformation and flow of matter and, as far as the preset invention is concerned, it is an analyzing method in which the pulse wave data are applied to an artery as a tube having an elasticity so as to derive a parameter that has not been obtained so far.

The “effective circulating efficiency (ECV)” will be explained by reference to a concrete calculating method, described later, developed by the Inventor.

The “Young's modulus (E)” is one of parameters indicative of an elasticity of an artery, and is given by a concrete calculating method, described later.

Next, there will be described a pulse wave data analyzing method developed by the Inventor.

Non-invasive, accurate, easy, and quick evaluation of functions of the greater circulatory system including the heart has been needed not only clinically but also publicly. To meet the need, the Inventor has succeeded in deriving calculating formulas that provide many useful function evaluation indexes, by using a pulse wave measured by a pulse wave measuring device (e.g., an apparatus 10 that will be described as an embodiment of the present invention) and applying a rheological, systematic analysis to the pulse wave. For example, the apparatus 10, described later, can concurrently measure respective blood pressure values of four limbs, and it provides stress-related information, e.g., the above-indicated ABI, PWV, etc. (this stress is a pressure wave produced when high-pressure blood is outputted into the aorta that is highly elastic). However, information related to a volume pulse wave as a strain (this strain is a change of a volume of the arterial system) is insufficient, and accordingly a pulse wave velocity cannot be evaluated in a satisfactory manner.

First of all, respective abbreviated symbols of function evaluation indexes described in the present specification are explained below.

1. Function evaluation indexes related to functions of the heart are stroke volume (ΔVst), cardiac output (CO), cardiac work (ECW), cardiac index (CI), and arterial circulatory efficiency (ACE).

2. Function evaluation indexes related to functions of the aortic system are main arterial system pulse wave velocity (Cm), main artery volume elasticity modulus (Km), main artery flow velocity (Um), main artery inner radius (Rᵢ), main artery wall thickness (h_{A}), main artery outer radius (Rₒ), Young's modulus (E_{A}), and effective circulating volume (ECV or Ve).

3. Function evaluation indexes related to functions of respective arteries of four limbs are pulse wave velocities (Cᵣᵤ, Cₗᵤ, Cᵣₗ, Cₗₗ: in the present specification, the left suffix attached to each function evaluation index related to the functions of the four limb arteries indicates a right limb (r) or a left limb (l), and the right suffix indicates an upper limb (u) or a lower limb (l) and accordingly the suffixes “ru”, “lu”, “rl” and “ll” indicate the right upper limb, the left upper limb, the right lower limb, and the left lower limb, respectively), artery flow velocities (Uᵣᵤ, Uₗᵤ, Uᵣₗ, Uₗₗ), mean pulse wave volumes (ΔVᵣᵤ, ΔVₗᵤ, ΔVᵣₗ, ΔVₗₗ), a total mean pulse wave volume (ΣΔV), artery inner radii (rᵢᵣᵤ, rᵢₗᵤ, rᵢᵣₗ, rᵢₗₗ), artery wall thickness values (hᵣᵤ, hₗᵤ, hᵣₗ, hₗₗ), artery outer radii (rₒᵣᵤ, rₒₗᵤ, rₒᵣₗ, rₒₗₗ), Young's modulus values (Eᵣᵤ, Eₗᵤ, Eᵣₗ, Eₗₗ), and artery minute flow rates (Frᵣᵤ, Frₗᵤ, Frᵣₗ, Frₗₗ).

The pulse wave velocity (Cm) is calculated by dividing a difference (l_{Aa}−l_{Ab}) of a distance (l_{Ab}) between an aortic valve (A) and an upper limb (brachia: b) and a distance (l_{Aa}) between the aortic valve (A) and a lower limb (ankle: a), by a time difference (Δt_{ba}) between respective rising points (respective feet: fb, fa) of respective volume pulse waves (see FIG. 2) measured at the upper and lower limbs, respectively. The pulse wave velocity (Cm) is a mathematical function of a volume elasticity modulus of an entire main arterial system (the main arterial system is so-called an elastic arterial system) consisting essentially of the aorta and first branches that are directly connected to the heart and have respective large inner volumes. Thus, the pulse wave velocity (Cm) needs to be dealt with as a function of a constant of a body, unlike pulse wave velocities measured from muscular arteries that are distributed in limbs' muscles and each have a simple shape, or a Young's modulus as a constant of a substance. It goes without saying that the main arterial system pulse wave velocity is influenced by the elastic characteristic of peripheral arteries. In addition, it is possible that the main arterial system pulse wave velocity be related to a physiological function that connects between the center and the periphery, and maintains and adjusts the systemic blood circulation. This is one of focal points of the present analyzing method.

In the case where a pulse wave is measured from a carotid artery, information related to the cerebral blood circulation can be obtained. In addition, when a portion of the data needed for actual calculations is missing, an available portion of the data can be used in place of the missing data (for example, in the case where pulse wave data corresponding to a left upper limb (lu) are missing, pulse wave data corresponding to a right upper limb (ru) can be used in place of the missing data.

Measurement and Analysis

A pressure-volume pulse wave starts with a time (A) when the aortic valve opens. The start point of propagation of the pulse wave is the time (A). The pulse wave velocity (Cm) that is calculated by dividing the difference (l_{ba}) of the distance (l_{Ab}) between the aortic valve (A) and the upper limb (brachia: b) and the distance (l_{Aa}) between the aortic valve (A) and the lower limb (ankle: a), by the time difference (Δt_{ba}) between the respective rising points (respective feet: fb, fa) of the respective volume pulse waves captured by respective cuffs worn on the upper and lower limbs, respectively, is a mathematical function of the volume elasticity modulus of the entirety main arterial system consisting essentially of the aorta and the first branches that are directly connected to the heart and whose inner volumes are large. It goes without saying that the main arterial system pulse wave velocity is influenced by the elastic characteristic of peripheral arteries. In addition, it is possible that the main arterial system pulse wave velocity be related to a physiological function that connects between the center and the periphery and maintains and adjusts the systemic blood circulation. This is a focal point of the present analyzing method. It is not easy to detect the time A when blood is ejected by the contraction of the left ventricle and the systemic blood circulation starts. If a time period between a time point Q on an electrocardiogram when an electric signal stimulating the cardiac contraction occurs, and the time A can be measured, measurements and analyses can be easily performed. In order to obtain the main arterial system pulse wave velocity as an average pulse wave velocity that represents the great main arterial system including the aorta as its central portion, the velocity needs to satisfies the following formula, though this satisfaction is not a sufficient condition but a necessary condition:
Cm=l_{ba}/Δt_{ba}=l_{Aa}/Δt_{Aa}=l_{Ab}/Δt_{Ab}
where l_{ba}=l_{Aa}−l_{Ab}−Δt_{ba}=Δt_{Aa}−Δt_{Ab},
Δt_{Aa}=(Δt_{Qa}−Δt_{QA}), and Δt_{Ab}=(Δt_{Qb}−Δt_{QA}) (Formula 1)

According to Formula 1, the time period QA (Δt_{QA}) is calculated, as follows:
Δt_{QA}=(I_{ba}Δt_{Qb}−l_{Ab}Δt_{ba})l_{ba} (Formula 2)

It is assumed that a time when the aorta closes corresponds to a sound II on a phonocardiogram (PCG) recorded concurrently with the ECG, and if a time period Q-II (Δt_{Q-II}) is thus obtained:
Δt_{Q-II}−Δt_{QA}=ET (Formula 3)

According to Formula 3, an ejection time (ET) can be obtained.

Analysis of Main Arterial System Pulse Wave Velocity

The stress that the high-pressure blood is outputted into the highly elastic aorta causes the strain that the volume of the main arterial system changes. Hill's formula (Formula 4) assuming the relationship between the stress and the strain, i.e., that volume elasticity modulus determines pulse wave velocity is applied:
Cm=(V/ρ(∂P/∂V))^{1/2} (Formula 4)
where ρ is a density and V is a volume.

Though the volume V is a mother that produces a volume change, Hill does not give a special meaning to the volume V. However, the Inventor has found that when Formula 4 is applied to the main arterial system pulse wave velocity, the volume V has an important physiological meaning. Since the concept of this volume is near to effective circulating volume (ECV), this volume is called Ve so as to be distinguished from a common volume V. Concerning the ECV, for example, a book of physiology, written by Berne and Levy, only gives the following description: ECV is not a measurable specific fraction of the body fluid, but it can reflect appropriateness of return from the tissues. That is, ECV is related to degree of filling, and pressure, of the vascular system. Thus, they only introduce the concept of ECV that is not concrete.

Hence, the concrete features of the volume Ve, found by the Inventor, are described below. That is, the volume ECV (Ve) as a new index has, e.g., the following features (1) through (9):

(1) This index can be calculated as an actual value, under conditions that (a) a pulse wave velocity (Cm) is measured and (b) the ratio of stress to strain (∂P/∂V) is obtained.

(2) Normal values of this index vary depending on ages. However, it is useful to compare this index with a total circulating arterial blood volume (a total blood volume is about 7% of a body weight, and the arterial blood volume is about 19% of the total blood volume).

(3) This index Ve is increased in some hypertensive patients, and decreased in other hypertensive patients. Thus, the index Ve is information useful in making a diagnosis or determining a treatment.

(4) The index Ve is significantly increased in heart-failure patients or aged persons. Thus, this index is information essential in making a prognosis.

(5) This index is related to polypeptides (ANP, BNP) that operate in controlling a water content.

(6) This index can be used to evaluate cardiac functions quickly, by being compared with a sum (ΣΔV) of respective changes of volumes of arteries during one heartbeat.

(7) According to Formula 4, this index can be used to calculate a pulse wave velocity (Cp) of a peripheral artery, if a ratio of stress to strain is obtained from a local portion of the peripheral artery.

(8) A Young's modulus (E) as a constant of a substance can be calculated based on the pulse wave velocity derived from the volume elasticity modulus as a constant of a body, while inner and outer radii, and a wall thickness value, of the peripheral artery are used.

(9) A degree of arteriosclerosis can be known from respective Young's modulus values (Eᵣᵤ, Eₗᵤ, Eᵣₗ, Eₗₗ) of respective arteries of four limbs.

Next, there will be explained a method of calculating, based on a height (ΔA) of the volume pulse wave, an internal-pressure change (ΔAp), a mean internal-pressure change (ΔApₘₑₐₙ), and a mean volume change (ΔVₘₑₐₙ). The following Formulas 5, 6, and 7 can be used to calculate, based on the chart shown in FIG. 2, the parameters ΔAp, ΔApₘₑₐₙ, ΔVₘₑₐₙ.
ΔA×α=ΔAp (Formula 5)
ΔAp×% MAP=ΔApₘₑₐₙ (Formula 6)
ΔAPₘₑₐₙ×γ/(ΔApₘₑₐₙ±ε)=ΔVₘₑₐₙ (Formula 7)

In Formula 5, ΔA indicates a height (millimeters) of the wave drawn on the chart; and a indicates a parameter that corrects the ratio of an actual pressure to the wave height on the chart (for example, when 50 mmHg corresponds to 18 mm on the chart, α=50/18). In Formula 6, % MAP is a percentage of a mean arterial pressure with respect to the wave height, under a assumption that the volume pulse wave is treated as a waveform of blood pressure. In Formula 7, γ and ε are correction factors that are used to convert a change of an internal pressure of a cuff into a change of a volume of the cuff according to Boyle's law, and mean that an inflation of γ ml is needed to obtain an internal pressure of the cuff, i.e., a counter pressure of ε mmHg. In each cuff, a control device of a pressure sensor that is connected to a quantitative air supply device is needed (for example, when 200 ml of air is supplied and a counter pressure of 60 mmHg is obtained, γ=200 and ε=60).

Next, there will be explained a method of calculating a strain ΔVp corresponding to a systolic pressure (Ps). The strain ΔVp can be calculated according to the following Formula 8:
ΔAp×γ/(ΔAp+ε)=ΔVp (Formula 8)

Respective strain values corresponding to four limbs, i.e., a right upper limb (ru), a left upper limb (lu), a right lower limb (rl), and a left lower limb (ll) can be calculated.

Blood pressure values as stresses, and their abbreviations are as follows: a systolic blood pressure (Ps), a mean blood pressure (Pm), a diastolic blood pressure (Pd), a pulse pressure (PP), a main mean blood pressure (harmonic mean: mPm=4/(1/Pmᵣᵤ+1/Pmₗᵤ+1/Pmᵣₗ+1/Pmₗₗ)), a mean pulse pressure (harmonic mean: PPm=4/(1/PPᵣᵤ+1/PPₗᵤ+1/PPᵣₗ+1/PPₗₗ)), and a mean diastolic blood pressure (harmonic mean: Pdm=4/(1/Pdᵣᵤ+1/Pdₗᵤ+1/Pdᵣₗ+1/Pdₗₗ)). Since a pulse wave velocity of a main artery is a harmonic mean of respective pulse wave velocities of respective segments of the main artery, all mean values are calculated as harmonic mean values throughout the present analysis.

Next, there will be explained a method of calculating a stroke volume (ΔVstₘₑₐₙ). The volume of blood outputted during one cardiac cycle (cc) causes the change of volume of all the arteries, and a major portion of this change can be captured at the four limbs. Since a volume change calculated according to Formula 7 is substantially simultaneously captured at the four limbs, a total mean pulse wave volume (ΣΔVₘₑₐₙ) can be calculated by summing up all those volume changes.
ΣΔVₘₑₐₙ=ΔVᵣᵤ₋ₘₑₐₙ+ΔVₗᵤ₋ₘₑₐₙ+ΔVᵣₗ₋ₘₑₐₙ+ΔVₗₗ₋ₘₑₐₙ (Formula 9)

The total mean pulse wave volume can be regarded as the amount of blood that is driven in the arterial system as a whole by pulse waves that are simultaneously produced by the mass of blood outputted during one heartbeat. However, the outputting of blood does not continue throughout one cardiac cycle, but it is limited to within the ejection time (ET). Therefore, the stroke volume is given according to the following Formula 10:
ΔVstₘₑₐₙ=ΣΔVₘₑₐₙ×ET/cc (Formula 10)

In addition, a cardiac output (CO), a cardiac index (CI) per body surface area (BSA), and an external cardiac work (ECW) can be calculated according to the following Formulas 11, 12, and 13:
CO=ΔVstₘₑₐₙ×60/cc (Formula 11)
where 60/cc is a number of heartbeats per minute.
   CI═CO/BSA (Formula 12)
   ECW=mPmΔVstₘₑₐₙ+ρΔVstₘₑₐₙU²m/2 (Formula 13)

In Formula 13, Um is a main artery mean flow velocity, and can be calculated according to the following Formula 14:
Um=PPm/ρCm (Formula 14)

In this stage, the new index ECV (Ve) can be calculated.
Ve=ρC²m·ΔVstₘₑₐₙ/mPm (this formula corresponds to formula A according to the present invention) (Formula 15)

Assuming that generally a total blood volume is 7% of a body weight and an arterial blood volume (ABV) is 19% of the total blood volume, the arterial blood volume can be compared with the absolute value of the index Ve. The ratio of the dynamic index ΣΔVₘₑₐₙ to the static index Ve is a cardiac-function evaluation index called an arterial circulatory efficiency (ACE). The arterial circulatory efficiency ACE is a new index that indicates what proportion of the arterial blood filling the arteries, giving tension to the walls of the arteries, and supporting the mean blood pressure can be exchanged during one heartbeat. Conventionally there have been used cardiac-function evaluation indexes obtained on the side of the heart; such as CO, CI, ejection fraction, etc. In contrast, the arterial circulatory efficiency ACE developed by the Inventor is a sensitive and novel cardiac-function evaluation index obtained on the side of effector organ.

A total blood flow rate (Q) used to calculate a viscosity is given by dividing a sum of the volume Ve (Formula 15) and the total mean pulse wave volume (ΣΔV), by the ejection time (ET).
Q=(Ve+ΣΔV)/ET (Formula 16)

Assuming that the aorta is a cylinder, an inner radius (R) {i.e., an inner radius (R_{Di}) when being passively dilated} of the aorta, and a change (ΔRᵢ) of the inner radius are obtained. If the blood of the stroke volume (ΔVstₘₑₐₙ) fills the cylinder at the mean flow velocity (Um) within the ejection time (ET), the following formula is obtained:
ΔVstₘₑₐₙ=πR²_{Di}·Um·ET (Formula 17)

Therefore, the inner radius is given by the following Formula 18:
R_{Di}=(ΔVst/πUm·ET)^{1/2} (Formula 18)

In addition, an inner radius (R_{Ci}) when being passively contracted is given by the following Formula 19:
R_{Ci}=2R_{Di}Cm/(2Cm+Um) (Formula 19)

Moreover, the change (ΔR) of the inner radius is given by the following Formula 20:
2ΔRᵢ=Rᵢ·Um/Cm (Formula 20)

To calculate a wall thickness (h) of an artery, a relationship between respective changes of inner and outer volumes of the artery is needed. An article the first author of which is the Inventor (Nakayama, R. et al: A theoretical approach to the volume pulse wave, Am. Heart J. 86. 96-106 (1973)) describes the following formula representing a relationship regarding a volume pulse wave of a limb or a peripheral portion:
ΔVₒₜ=κ{Vₒ·Uₜ/C}
where κ is a proportion constant.

An artery's outer volume (Vₒ) corresponding to a change (ΔVₒₜ) thereof at a time t is a diastolic volume; and a change (ΔVᵢₜ) of an artery's inner volume corresponds, according to Formula X, to a systolic artery's inner volume (Vᵢ), as follows:
ΔVᵢₜ=κ{Vᵢ·Uₜ/C} (Formula X′)

If it is assumed that β=the ratio (κ) of Formula X to Formula X′, the following formula is obtained:
β=ΔVₒₜ/ΔVᵢₜ=Vₒ/Vᵢ (Formula 21)

If an artery's volume (Vmi) corresponding to the mean blood pressure (mPm) is replaced with a model of a cylinder having an inner radius (Ri) and a length (L), and an artery's volume (Vdo) corresponding to the mean diastolic blood pressure (Pdm) is replaced with a model of a cylinder having an outer radius (Ro) and a length (L), the ratio of the artery's outer volume to the artery's inner volume can be used to calculate the ratio (β) of a square of the outer radius to a square of the inner radius.

$$\begin{matrix}Vdo / Vmi = ( ρ {Cm}^{2} Δ Vst / Pdm ) / ( ρ {Cm}^{2} Δ Vst / mPm ) \\ = mPm / Pdm \\ = {Ro}^{2} / {Ri}^{2} \\ = β\end{matrix} \qquad ( Formula 22 )$$

Here, though the stroke volume ΔVst is ejected during the systole, it is assumed that the stroke volume ΔVst as a strain does not change between the systole and the diastole.

On the above-indicated model, a wall thickness (h_{A}) of the cylindrical aorta is equal to a difference of the outer radius (Ro), and the inner radius (Ri), of the artery.
h_{A}=Ro−Ri=Ri(Ro/Ri)−Ri=ωRi(β^{1/2}−1) (Formula 23)
where ω is an integer as a correction factor.

Here, the correction factor ω is selected from one or two, such that the selected integer more appropriately satisfies a conventionally known relationship represented by the following formula: h/2Ri≈0.08. Thus, the correction factor ω corrects distortion.

Therefore, the outer radius (R_{C0}) of the aorta is given by the following Formula 24:
R_{C0}=R_{Ci}+h_{A} (Formula 24)

A constant of a body that indicates an elastic characteristic of the main arterial system including the aorta, and a volume elasticity modulus (Km) are obtained, based on Hill's formula (Formula 4), according to the following Formula 25:
Km=ρC²m (Formula 25)

Since the inner and outer radii and wall thickness of the aorta have been obtained, a Young's modulus (E_{A}) that could be called a constant of a substance that indicates an elastic characteristic can be obtained, based on Moens-Korteweg's formula, according to the following Formula 26:
E_{A}=2ρR_{Co}·C²m/h_{A} (this formula corresponds to formula B according to the present invention) (Formula 26)

Since the flow velocity and the inner radius of the artery have been obtained, this system is completed by the calculation of a flow rate. When a blood flow rate of an arterial system that is too complicated is calculated, it does not suffice to use a simple cylinder model and obtain an instantaneous flow rate and respective cross-section areas of some segments of the arterial system. Hence, a blood viscosity (μ) is calculated, according to Poiseuille's formula, based on a total blood flow rate (Q: Formula 16) of the arterial system as a whole that is driven as a linear steep rise of an upper-limb pulse wave that is near to a steady flow, i.e., a reverse pressure gradient (−1/Cm·∂P/∂t):
Q=(ΣΔV+Ve)/ET (Formula 16)
μ={πRᵢ⁴m/Q}·{⅛}·{1/Cm·∂P/∂t} (Formula 27)

Since a kinematic viscosity (Λ) can be calculated based on the blood viscosity and density, a Reynolds number (Re) can be calculated, according to the following Formula 28, based on the artery's inner radius and the flow velocity:
Re=2Ri·Um/Λ (Formula 28)

The blood viscosity that shows a stable value is used to calculate a blood flow rate of the peripheral arterial system, and is also used to judge whether the results obtained according to the present method are acceptable or appropriate. In the calculation according to Formula 27, the mean artery's inner radius (Rim) is replaced with the inner radius Rci (Formula 19). However, some correction may be needed. More specifically explained, a correction according to the following formula: Rim=κRci (κ≈from 1.0 to 0.6) may be needed.
Cᵣᵤ={PsᵣᵤVe/ρΔVpᵣᵤ}^{1/2}
Cₗᵤ={PsₗᵤVe/ρΔVpₗᵤ}^{1/2}
Cᵣₗ={PsᵣₗVe/ρΔVpᵣₗ}^{1/2}
Cₗₗ={PsₗₗVe/ρΔVpₗₗ}^{1/2} (Formulas 29)

Mean flow velocities are calculated based on the velocities PWV given by the above-indicated Formulas 29, and Allievi's formula.
Umᵣᵤ=PPᵣᵤ/ρCᵣᵤ
Umₗᵤ=PPₗᵤ/ρCₗᵤ
Umᵣₗ=PPᵣₗ/ρCᵣₗ
Umₗₗ=PPₗₗ/ρCₗₗ (Formulas 30)

Since the pulse wave velocities (C) and the flow velocities (U) have been obtained, the ratio of a change (Δrₒ) of an artery's outer diameter to the artery's outer diameter (rₒ), by applying Formula 20 to a peripheral artery.
2Δrₒ/rₒ=U/C (Formula 31)

A mean volume change (ΔVₘₑₐₙ) is defined by a change of a cross-section area that moves at the pulse wave velocity (C) during one cardiac cycle.
ΔVₘₑₐₙ=πcc·C{(rₒ+Δrₒ)²−r²ₒ} (Formula 32)

Based on Formulas 31 and 32, a radius of the artery's outer diameter (Δrₒ) is calculated according to the following Formula 33, and the artery's outer diameter (rₒ) is calculated, based on the obtained value Δrₒ, according to Formula 31.
Δrₒ={ΔVₘₑₐₙ·U/πcc(4C²+C·U)}^{1/2} (Formula 33)
rₒ=2Δrₒ·C/U (Formula 31′)

A wall thickness (h) of the cylindrical peripheral artery is given as a difference of the artery's outer radius (rₒ) and the artery's inner radius (rᵢ). Therefore, Formula 23 is used. Since, however, the artery's outer radius (rₒ) is first given, not β(=rₒ/rᵢ) but 1/β(=rᵢ/rₒ) is used.

$$\begin{matrix}{h}_{ru} = {r}_{C0ru} \{ 1 - {( Pdm / mPm )}^{1 / 2} \} \\ = {r}_{C0ru} \{ 1 - {( 1 / β )}^{1 / 2} \} \\ {h}_{lu} = {r}_{C0lu} \{ 1 - {( 1 / β )}^{1 / 2} \} \\ {h}_{rl} = {r}_{C0rl} \{ 1 - {( 1 / β )}^{1 / 2} \} \\ {h}_{ll} = {r}_{C0ll} \{ 1 - {( 1 / β )}^{1 / 2} \}\end{matrix} \qquad ( Formulas 34 )$$

1/βᵣᵤ=Ve·Uᵣᵤ/ΔV_{Pru}·Cᵣᵤ
1/βₗᵤ=Ve·Uₗᵤ/ΔV_{Plu}·Cₗᵤ
1/βᵣₗ=Ve·Uᵣₗ/ΔV_{Prl}·Cᵣₗ
1/βₗₗ=Ve·Uₗₗ/ΔV_{Pll}·Cₗₗ (Formulas 35)

From the above-indicated results, an artery's elastic modulus that is deeply related to arteriosclerosis, i.e., a Young's modulus can be calculated according to Formulas 36.
Eᵣᵤ=2ρrₒᵣᵤC²ᵣᵤ/hᵣᵤ
Eₗᵤ=2ρrₒₗᵤC²ₗᵤ/hₗᵤ
Eᵣₗ=2ρrₒᵣₗC²ᵣₗ/hᵣₗ
Eₗₗ=2ρrₒₗₗC²ₗₗ/hₗₗ (these formulas correspond to formulas C according to the present invention) (Formulas 36)

If it is assumed that a peripheral artery has an elongate cylindrical shape, an inner diameter of the artery can be calculated, based on the outer diameter and the wall thickness, according to the following Formulas 37. In addition, a cross-section area of the artery is calculated, and a flow rate can be calculated based on the cross-section area and a known systolic mean flow velocity.
r_{doru}−hᵣᵤ=r_{ciru}
r_{dolu}−hₗᵤ=r_{cilu}
r_{dorl}−hᵣₗ=r_{cirl}
r_{doll}−hₗₗ=r_{cill} (Formulas 37)

According to the present, limb artery's blood flow rate measuring method, a flow rate (FR) is calculated, based on the blood viscosity (μ) obtained according to Formula 27, the pressure gradient indicated by the decreasing portion of the volume pulse wave that corresponds to the diastole of the heart, the radius (ri), and Poiseuille's formula, according to the following Formulas 38:
FRᵣᵤ={π(rᵢᵣᵤ)⁴/μ}·{⅛}·{1/Cᵣᵤ·∂P/∂t}
FRₗᵤ={π(rᵢₗᵤ)⁴/μ}·{⅛}·{1/Cₗᵤ·∂P/∂t}
FRᵣₗ={π(rᵢᵣₗ)⁴/μ}·{⅛}·{1/Cᵣₗ·∂P/∂t}
FRₗₗ={π(rᵢₗₗ)⁴/μ}·{⅛}·{1/Cₗₗ·∂P/∂t}

### BRIEF DESCRIPTION OF THE DRAWINGS

FIG. 1 is a diagrammatic view for explaining a construction of an upper limb and lower limb blood pressure measuring apparatus. In this figure, reference numerals 10, 16 and 18, 70, and 71 designate the upper limb and lower limb blood pressure measuring apparatus, blood pressure measuring devices, an action potential measuring device, and a heart sound measuring device, respectively.

FIG. 2 is a view of a chart showing an electrocardiogram, a phonocardiogram, and changes of blood pressure.

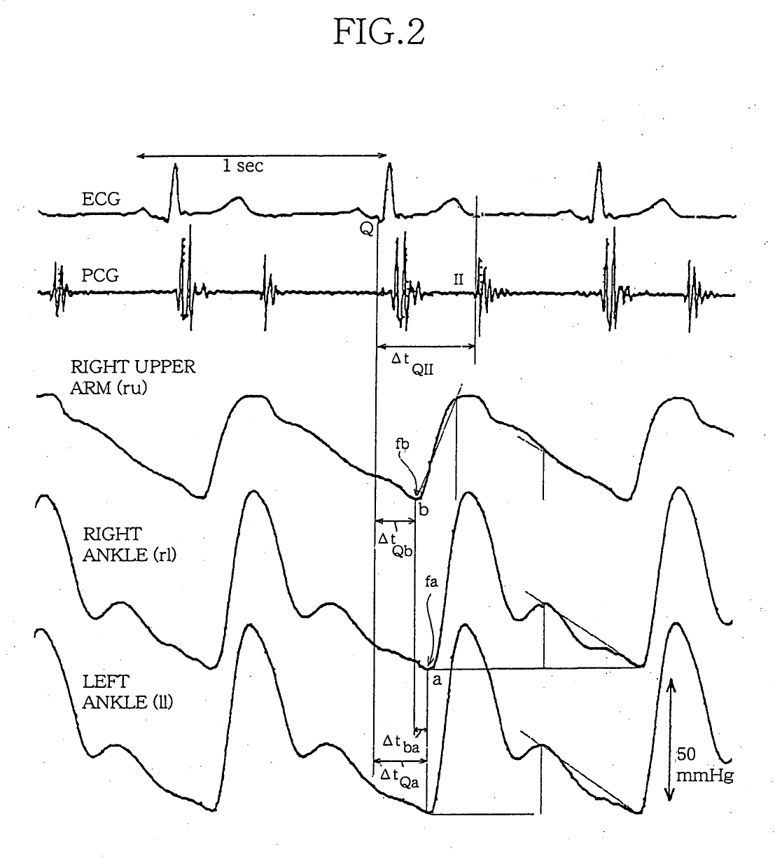

FIG. 3 is a flow chart representing a software as an embodiment of the present invention.

### BEST MODE FOR CARRYING OUT THE INVENTION

Hereinafter, there will be described an embodiment of the present invention in detail by reference to the drawings. However, the technical scope of the present invention is not limited by the below-described embodiment, and the present invention may be embodied with various changes without departing from the spirit thereof. In addition, the technical scope of the present invention encompasses equivalents of the invention.

Construction of Pulse Wave Measuring Device

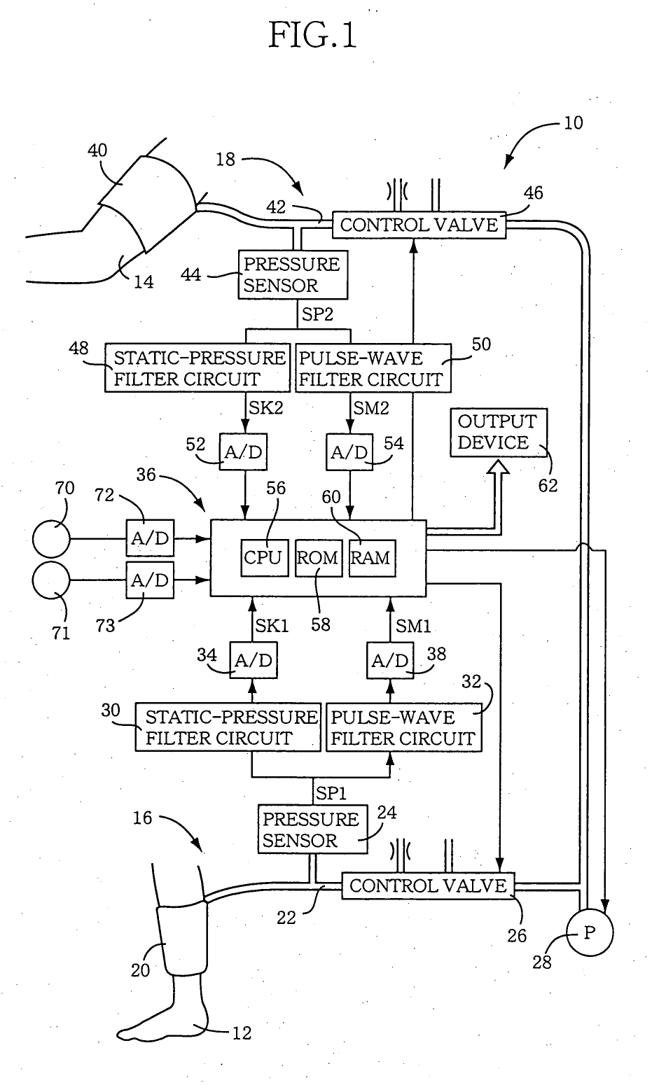

The present invention may be applied to, e.g., an apparatus disclosed by Japanese Patent Application Publication No. 2001-340306. FIG. 1 is a diagrammatic view for explaining a construction of a blood pressure measuring apparatus 10 (hereinafter, referred to as the apparatus 10) for measuring blood pressure values of a lower limb and an upper limb.

The apparatus 10 shown in FIG. 1 is adapted such that an ankle 12 is selected as the lower limb and an upper arm 14 is selected as the upper limb. The apparatus 10 performs a measuring operation, in a state in which a living subject takes a face-down position, a lateral position, or a lateral position.

In FIG. 1, the apparatus 10 includes an ankle blood pressure measuring device 16 that timewise measures a blood pressure of the ankle 12 (e.g., a right ankle: preferably, two cuffs 20 are employed for left and right ankles, respectively, though not shown); and an upper arm blood pressure measuring device 18 that timewise measures a blood pressure of the upper arm 14. The ankle blood pressure measuring device 16 includes a cuff 20 that includes a belt-like cloth bag and a rubber bag accommodated in the cloth bag and is adapted to be wound around the ankle 12 of the subject; a pressure sensor 24 that is connected to the cuff 20 via a pipe 22; a control valve 26; and an air pump 28. The control valve 26 is switchable to three positions, i.e., a pressure supply position where the control valve 26 allows a pressure to be supplied to the cuff 20; a slow deflation position where a degree of opening of the electric valve is changed to allow the pressure to be slowly discharged from the cuff 20; and a quick deflation position where the electric valve allows the pressure to be quickly discharged from the cuff 20.

The pressure sensor 24 detects the pressure in the cuff 20, and supplies a pressure signal SP1 representing the detected pressure, to a static-pressure filter circuit 30 and a pulse-wave filter circuit 32. The static-pressure filter circuit 30 includes a low-pass filter, and extracts, from the pressure signal SP1, a static component of the detected pressure, i.e., a cuff pressure signal SK1 representing a cuff pressure PC1, and supplies the cuff pressure signal SK1 to an electronic control device 36 via an A/D converter 34.

The pulse-wave filter circuit 32 includes a band-pass filter, and extracts, from the pressure signal SP1, an oscillatory component of the detected pressure that has certain frequencies, i.e., a pulse wave signal SM1, and supplies the pulse wave signal SM1 to the control device 36 via an A/D converter 38. Since the pulse wave signal SM1 represents an ankle pulse wave ML occurring from arteries (mainly, a posterior tibial artery) of the ankle 12 that are pressed by the cuff 20, the pulse-wave filter circuit 32 functions as a lower limb pulse wave detecting device.

The upper arm blood pressure measuring device 18 includes a cuff 40 (preferably, two cuffs 40 are employed for left and right upper arms, respectively, though not shown); a pipe 42, a pressure sensor 44, and a control valve 46 that have respective constructions identical with those of the counterparts of the ankle blood pressure measuring device 16. During the measuring operation, the cuff 40 is wound around the upper arm 14, and the control valve 46 is connected to the air pump 28. The pressure sensor 44 detects a pressure in the cuff 40, and supplies a pressure signal SP2 representing the detected pressure, to a static-pressure filter circuit 48 and a pulse-wave filter circuit 50 (the two circuits 48, 50 have respective constructions identical with those of the counterparts of the ankle blood pressure measuring device 16). The static-pressure filter circuit 48 extracts, from the pressure signal SP2, a static component of the detected pressure, i.e., a cuff pressure signal SK2 representing a cuff pressure PC2, and supplies the cuff pressure signal SK2 to the electronic control device 36 via an A/D converter 52. The pulse-wave filter circuit 50 extracts, from the pressure signal SP2, an oscillatory component of the detected pressure that has certain frequencies, i.e., a pulse wave signal SM2, and supplies the pulse wave signal SM2 to the control device 36 via an A/D converter 54.

In addition, the apparatus 10 includes an action-potential measuring device 70 that can measure an action potential of the heart (based on data obtained by this device, an electrocardiogram can be drawn); and a heart-sound measuring device 71 that can measure a heart sound. These devices 70, 71 supplies respective signals to the control device 36 via respective A/D converters 72, 73.

The electronic control device 36 is constituted by a microcomputer including a CPU 56, a ROM 58, a RAM 60, and an I/O port, not shown. The control device 36 processes signals according to programs pre-stored in the CPU 56 or the ROM 58, while utilizing a data storing function of the RAM 60, and outputs, from the I/O port, respective drive signals to the air pump 28 and the two control valves 26, 46, and controls what is outputted by an output device 62. The output device 62 includes, for example, a pen recorder, a monitor, and an appropriate recording medium (e.g., a hard disc, MO, FD, or CD).

Construction of Computer

Next, there will be described a construction of a computer that is used to implement a software in accordance with the present invention that will be described later.

### First Embodiment

First, this computer may be provided by the electronic control device 36 of the above-described apparatus 10. More specifically described, since the control device 36 is timewise supplied with the pulse wave data from the upper and lower limbs, the electrocardiogram data, and the phonocardiogram data, the control device 36 can concurrently process those data and calculate the parameters concerning the arteries.

### Second Embodiment

Alternatively, the above computer may be one that can read digital data outputted, and recorded on the recording medium, by the output device 62, and process those data. In this case, a common computer may be used to read the digital data from the recording medium and calculate, based on the data, the parameters concerning the arteries.

Algorithm of Software

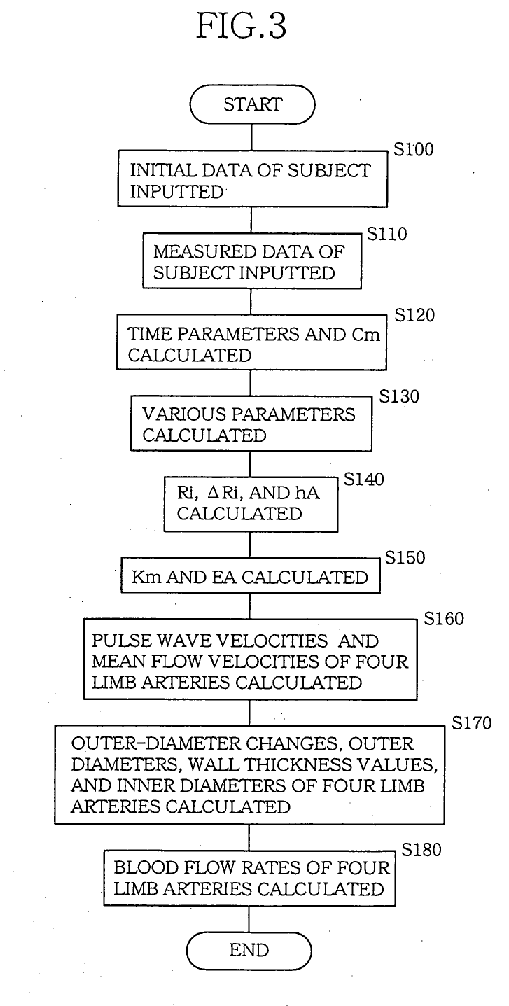

Next, there will be described an example of an algorithm that can implement the above-described calculating methods, by reference to FIG. 3.

First, initial data concerning a living subject (including, e.g., a stature, a body weight, a body surface area, a circulating blood volume, an arterial blood volume, l_{Ab}, l_{Aa}, and l_{ba}) are inputted (S100).

Subsequently, the measured data concerning the heart and the arteries are inputted based on the pulse waves, the electrocardiogram, and the phonocardiogram (S110). Those data may be inputted (1) while the apparatus 10 performs the measuring operation, or (2) based on the data already measured by the apparatus 10 (e.g., electronically recorded digital data, or data recorded on a chart sheet). That is, the term “input” encompasses not only a case where a computer recognizes, according to a pre-determined procedure, an appropriate point of time and automatically input data, but also a case where a human being reads data drawn on a chart sheet and manually inputs the data.

Then, based on the thus inputted data, time parameters, i.e., a cardiac cycle (cc), Δt_{Aa}, Δt_{Ab}, Δt_{ba}, Δt_{Aa}, Δt_{Qa}, Δt_{Q-II}, and an ejection time (ET), and a main arterial system pulse wave velocity (Cm) are calculated (S120).

Subsequently, ΣΔVₘₑₐₙ, ΔVstₘₑₐₙ, and Um are calculated, and various parameters, i.e., Ve, Ve/arterial blood volume, Ve/ΣΔVₘₑₐₙ, CO, an CI are calculated (S130).

In addition, Rᵢ, ΔRᵢ, and an aorta's wall thickness (h_{A}) are calculated (S140).

Then, a volume elasticity modulus (Km) and a Young's modulus (E_{A}) are calculated (S150).

In addition, four limb arteries' pulse wave velocities (Cᵣᵤ, Cₗᵤ, Cᵣₗ, Cₗₗ) and mean flow velocities (Uₘᵣᵤ, Uₘₗᵤ, Uₘᵣₗ, Uₘₗₗ) are calculated (S160).

Subsequently, based on four limb arteries' outer-diameter changes (Δrₒᵣᵤ, Δrₒₗᵤ, Δrₒᵣₗ, Δrₒₗₗ), outer diameters (rₒᵣᵤ, rₒₗᵤ, rₒᵣₗ, rₒₗₗ), wall thickness values (hᵣᵤ, hₗᵤ, hᵣₗ, hₗₗ), and inner diameters (rᵢᵣᵤ, rᵢₗᵤ, rᵢᵣₗ, rᵢₗₗ) are calculated (S170).

Then, respective blood flow rates (Frᵣᵤ, Frₗᵤ, Frᵣₗ, Frₗₗ) of the four limbs are calculated (S180).

In the above-indicated algorithm, if a plurality of parameters calculated in a plurality of steps, respectively, do not depend on each other, the order of those steps in the algorithm may be changed.

In addition, each value can be calculated by an appropriate one of the above-described calculating methods etc.

### ACTUAL MEASUREMENT EXAMPLE 1

Next, function evaluation indexes of an actual living subject are calculated using the above-described formulas.

Case 1

She is a nineteen-year-old, active and healthy high-school girl. She showed the following values: mPm=73 mmHg; PPm=48 mmHg; heart rate=67 b/min; Cm=504 cm/sec; Um=120 cm/sec; Ve=204 ml; ΣΔV=233 ml; Vst=74 ml; Co=4.9 L/min; CI=3.4 L/min/m²; Rci=0.701 cm; h_{A}=0.107 cm; total blood flow rate increase Q=1369 ml/sec (The value (FRₗᵤ) of the left upper limb, substituted by the value (FRᵣᵤ) of the right upper limb, was added. This applies to the following values, if appropriate); FRᵣᵤ=1196 ml/min; FRᵣₗ=1098 ml/min; and FRₗₗ=783 ml/min. A total blood flow rate of the four limb arteries was 4273 ml/min; E_{A}=4.05×10⁵ Nm⁻²; and respective Young's modulus values of the four limb arteries were such that Eᵣᵤ=5.91×10⁵ Nm⁻², Eᵣₗ=4.75×10⁵ Nm⁻², and Eₗₗ=3.82×10⁵ Nm⁻². The measured and calculated values, obtained from this case, are treated as standard values, and are indicated in parentheses for comparison with the values obtained from the other cases.

Case 2

She was an eighty-five-year-old, stretcher patient complaining of intense pains in the chest and back. However, ECG showed no signs of acute myocardial infarction. With mPm=122 mmHg (73) and PPm=105 mmHg (48), she was hypertensive. In addition, she showed: heart rate=56 b/min (67); Cm=1692 cm/sec (504); Um=78 cm/sec (120); Ve=1153 ml (204); ΣΔV=346 ml (233); Vst=62 ml (74); Co=3.5 L/min (4.9); and CI=2.6 L/min/m² (3.4). Thus, the cardiac output was slightly low, but it is notable that Ve is extremely high. This means that the arterial system as a whole, in particular, the aortic system has an enlarged inner lumen. Hence, the artery's diameters and wall thickness values were observed.

With Rci=1.111 cm (0.701); h_{A}=0.287 cm (0.107); and total blood flow rate increase Q=7648 ml/sec (1369), she showed: FRᵣᵤ=2220 ml/min (1196); FRᵣₗ=2100 ml/min (1098); and FRₗₗ=1626 ml/min (783). Thus, the respective blood flow rates of the four limb arteries were high. A total blood flow rate of the four limb arteries, 8166 ml/min (4273), was significantly high. Naturally, a degree of arteriosclerosis of the aorta was high to such an extent that E_{A}=29.4×10⁵ Nm⁻² (4.05), and respective Young's modulus values of the four limb arteries were considerably high to such an extent that Eᵣᵤ=24.73×10⁵ Nm⁻² (5.91), Eᵣₗ=20.59×10⁵ Nm⁻² (4.75), and Eₗₗ=24.17×10⁵ Nm⁻² (3.82). Aortic dissection is such a disease that blood that has invaded the aorta's wall via fissures of the aorta's endothelium, or the intrainterstitial bleeding fissures or dissects the wall, and thereby produces, in the wall, a pseudo-lumen that cooperates with the proper lumen to enlarge the artery's lumen and decrease the wall's thickness. Thus, the data obtained according to the present method reflect this disease.

Case 3

He was a seventy-three-year-old, company's president, and a high-spirited gentleman with moderate diabetes and hypertension under treatment. He showed: mPm=104 mmHg (73); PPm=67 mmHg (48); heart rate=66 b/min (67); Cm=1098 cm/sec (504); Um=77 cm/sec (120); Ve=780 ml (204); ΣΔV=266 ml (233); Vst=85 ml (74); Co=5.6 L/min (4.9); and CI=3.3 L/min/m² (3.4); Rci=1.091 cm (0.701); h_{A}=0.185 cm (0.107); and total blood flow rate increase Q=3789 ml/sec (1369). Thus, he showed changes corresponding to his age. A blood viscosity μ=0.044 poise (0.043), obtained as the ratio of the total blood flow rate to the pressure gradient, was normal. However, respective degrees of sclerosis of the aorta and the four limb arteries appear to be high. In addition, he showed: E_{A}=17.6×10⁵ Nm⁻² (4.05); Eᵣᵤ=22.0×10⁵ Nm⁻² (5.91); Eᵣₗ=16.8×10⁵ Nm⁻² (4.75), Eₗₗ=11.5×10⁵ Nm⁻² (3.82); FRᵣᵤ=926 ml/min (1196); FRᵣₗ=558 ml/min (1098); and FRₗₗ=360 ml/min (783). Thus, the respective blood flow rates of the four limb arteries were rather lower than those of CASE 1.

Case 4 (Self Measurement)

Next, the above-described calculations were applied to a person (the Inventor) having the following basic data: stature=172 cm; body weight=65 kg; body surface area=1.77 m²; estimated circulating blood volume=4.55 L; estimated arterial blood volume=865 ml (this volume is estimated based on the body weight); l_{Ab}=57 cm; l_{Aa}=120 cm; l_{ba}=63 cm; cardiac cycle (cc)=0.833 sec; Δt_{Qb}=0.18 sec; Δt_{Qa}=0.25 sec; Δt_{ba}=0.0694 sec; Δt_{QII}=0.417 sec; Δt_{QA} (=l_{ba}Δt_{Qb}−l_{Ab}Δt_{ba})=0.118 sec; ET (=Δt_{QII}−Δt_{QA})=0.299 sec; ET/cc=0.358 sec; PEP/ET=Δt_{QA}/ET=0.39; and Cm=63 cm/0.06944 sec=907 cm/sec.

Table I sums up the data.

TABLE 1

| | Pm | Ps/Pd | PP | Δ A | % | Δ Apₘₑₐₙ | Δ Ap × % | Pt | Δ Vₘₑₐₙ | Δ Vp |
|---|---|---|---|---|---|---|---|---|---|---|
| | mmHg | MmHg | mmHg | mm | MAP | mmHg | mmHg | mmHg | ml | ml |
| RU | 112 | 142/99 | 43 | 15 | 50 | 42 | 21 | +1 | 52 | 82 |
| LU | 114 | 151/100 | 51 | | | | | | | |
| RL | 125 | 174/98 | 76 | 26 | 41 | 72 | 30 | +4 | 67 | 109 |
| LL | 119 | 175/96 | 79 | 28 | 42 | 78 | 33 | +1 | 71 | 113 |
| | mPm | Pdm | PPm | | | | | | | |
| | 117 | 98 | 58 | | | | | | | |

Concerning LU (the left upper limb), missing data were substituted by the corresponding data obtained from RU (the right upper limb). Thus, ΣΔVₘₑₐₙ and ΔVstₘₑₐₙ were calculated as follows: ΣΔVₘₑₐₙ=52+52+67+71=242 (ml) and ΔVstₘₑₐₙ=242×0.358=87 (ml).

In addition, according to Formula 14, Um was calculated as follows: Um=PPm/ρCm=58×1333 dyn/cm²/1.056 g/cm³·907 m/sec=81 cm/sec.

According to Formula 15, Ve, Ve/arterial blood volume, and Ve/ΣΔVₘₑₐₙ were calculated as follows: Ve=1.056 g/cm³×(907 cm/sec)²×87 ml/117×1333 dyn/cm²=485 ml, Ve/arterial blood volume=485 ml/865 ml=0.56, and Ve/ΣΔVₘₑₐₙ=485 ml/242 ml=2.00. In addition, according to Formula 16, Q (=(ΣΔV+Ve)/ET=727/0.299)=2431 ml/sec, CO=6.24 L/min, and CI=3.52 L/min/m² were calculated.

In addition, according to Formula 18, R_{Di}=(87 ml/π·65 cm/sec·0.299 sec)^{1/2}=1.194 cm was calculated; and according to Formula 19, R_{Ci}=2×1.194 cm×907 cm/sec/(2×907 cm/sec+65 cm/sec)=1.156 cm, and ΔR (=R_{Di}·R_{Ci})=0.038 cm were calculated.

In addition, according to Formula 23, an aorta's wall thickness h_{A}=2×1.156 cm×(1.194^{1/2}−1)=0.2142 cm was calculated; and R_{co}=1.370 cm (Formula 24) was calculated.

In addition, according to Formulas 25 and 26, a volume elasticity modulus Km=0.87×10⁶ dyn/cm² and a Young's modulus E_{A}=11.11×10⁵ N/m² were calculated, respectively. According to Formula 27, a blood viscosity μ={π(1.156×0.8)⁴/2431}·{⅛}·{244×1333/907}=0.042 (poise) was calculated. In addition, a kinetic energy was ½×(ρ87×81²)=0.0301×10⁸ erg, and mPm×ΔVₛₜ=1.356×10⁸ erg, and therefore a total kinetic energy was 1.387×10⁸ erg (where ρ=1.056 g/ml was used).

According to Formulas 29, respective pulse wave velocities of the four limb arteries were calculated as follows:

$$\begin{matrix}{C}_{ru} = {( 142 \times 1333 dyn \text{/} {cm}^{2} \cdot 485 ml \text{/} 1.056 g \text{/} {cm}^{3} \cdot 82 ml )}^{1 / 2} \\ = 1029 cm \text{/} sec \\ {C}_{rl} = {( 174 \times 1333 dyn \text{/} {cm}^{2} \cdot 485 ml \text{/} 1.056 g \text{/} {cm}^{3} \cdot 109 ml )}^{1 / 2} \\ = 986 cm \text{/} sec \\ {C}_{ll} = {( 175 \times 1333 dyn \text{/} {cm}^{2} \cdot 485 ml \text{/} 1.056 g \text{/} {cm}^{3} \cdot 113 ml )}^{1 / 2} \\ = 974 cm \text{/} sec\end{matrix}$$

According to Formulas 30, respective mean flow velocities of the four limb arteries were calculated as follows:
Umᵣᵤ=43×1333 dyn/cm²/1.056 g/cm³·1029 cm/sec=53 cm/sec
Umᵣₗ=76×1333 dyn/cm²/1.056 g/cm³·989 cm/sec=97 cm/sec
Umₗₗ=79×1333 dyn/cm²/1.056 g/cm³·974 cm/sec=102 cm/sec

In view of Formulas 33 etc., respective outer-diameter changes (Δrₒ), respective outer diameters (rₒ), and respective wall thickness values (h) of the four limb arteries were calculated as follows: Concerning the right upper limb, Δrₒᵣᵤ={53 cm/sec×52 ml/π30/36(4×1029²+1029×53)}^{1/2}=0.0157 cm; rₒᵣᵤ=2×0.0157 cm×1029 cm/sec/53 cm/sec=0.6096 cm; hᵣᵤ=2×0.609 cm{1−(98/117)^{1/2}}=0.103 cm; and rᵢᵣᵤ=0.609−0.103=0.506 cm.

Concerning the right lower limb, Δrₒᵣₗ={97 cm/sec×67 ml/π30/36(4×989²+989×97)}^{1/2}=0.0249 cm; rₒᵣₗ=2×0.0249 cm×989 cm/sec/97 cm/sec=0.5076 cm; hᵣₗ=2×0.507 cm{1−(98/117)^{1/2}}=0.0859 cm; and rᵢᵣₗ=0.507-0.0859=0.421 cm.

Concerning the left lower limb, Δrₒₗₗ={102 cm/sec×67 ml/π30/36(4×948²+948×102)}^{1/2}=0.0274 cm; rₒₗₗ=2×0.0274 cm×948 cm/sec/102 cm/sec=0.509 cm; hₗₗ2×0.509 cm{1−(98/117)^{1/2}}=0.0863 cm; and rᵢₗₗ=0.509−0.0863=0.423 cm.

According to Formulas 38, respective blood flow rates of the four limbs were calculated as follows:

$$\begin{matrix}{Fr}_{ru} = \{ {π ( 0.402 cm )}^{4} \text{/} 0.0423 poise \} \cdot 1 / 8 \cdot 57.6 mmHg \text{/} sec \text{/} 1029 m \text{/} sec \\ = 18.08 ml \text{/} sec \\ = 1085 ml \text{/} min\end{matrix}$$

Frₗᵤ=(is treated as being equal to Frᵣᵤ)=1085 ml/min
Frᵣₗ={π(0.40 cm)⁴/0.0423 poise}·⅛·58.8 mmHg/sec/989 cm/sec=18.45 ml/sec=1107 ml/min
Frₗₗ={π(0.509 cm)⁴/0.0423 poise}·⅛·63.6 mmHg/sec/974 cm/sec=54.24 ml/sec=3254 ml/min
Thus, a total blood flow rate of the four limbs was calculated as 6531 ml/min.

In addition, according to Formulas 36, respective Young's modulus values of the four limb arteries were calculated as follows:
Eᵣᵤ=2×1.056×0.609×1029²/0.103=13.22×10⁵ Nm⁻²
Eᵣₗ=2×1.056×0.508×989²/0.0859=12.22×10⁵ Nm⁻²
Eₗₗ=2×1.056×0.509×974²/0.0863=11.82×10⁵ Nm⁻²

Thus, with the series of formulas developed by the Inventor, the new parameters (e.g., the volume elasticity modulus (Km) and the Young's modulus (E_{A})) can be calculated based on the pulse wave data. These parameters can be used to evaluate functions of a living being in a non-invasive, accurate, easy, and quick manner.

1. A pulse wave analyzing software characterized by applying a rheological analysis to pulse wave data measured at two or more different portions of a living being and thereby calculating at least one parameter selected from an effective circulating volume (ECV, Ve) and a Young's modulus (E).

2. The pulse wave analyzing software according to claim 1, wherein the effective circulating volume (ECV, Ve) is given by a following formula A:
Ve=ρC²m·ΔVstₘₑₐₙ/mPm Formula A
(where ρ is a density; Cm is a mean pulse wave velocity; ΔVstₘₑₐₙ is a stroke volume; and mPm is a main mean blood pressure),
and wherein the Young's modulus (E) is given by a following formula B or following formulas C:
E_{A}=2ρRₒ·C²m/h_{A} Formula B
(where E_{A} is a Young's modulus of an aorta; Rₒ is an outer radius of the artery; and h_{A} is a wall thickness of the artery),
Eᵣᵤ=2ρrₒᵣᵤC²ᵣᵤ/hᵣᵤ
Eₗᵤ=2ρrₒₗᵤC²ₗᵤ/hₗᵤ
Eᵣₗ=2ρrₒᵣₗC²ᵣₗ/hᵣₗ
Eₗₗ=2ρrₒₗₗC²ₗₗ/hₗₗ Formula C
(where suffixes “ru”, “lu”, “rl”, and “ll” mean a right upper limb, a left upper limb, a right lower limb, and a left lower limb, respectively; rₒ is an outer radius of an artery of a limb; C is a pulse wave velocity; and h is a wall thickness of the artery).

3. A computer capable of implementing the pulse wave analyzing software according to claim 1.

4. A pulse wave analyzing apparatus characterized by including a blood pressure measuring device capable of measuring pulse wave data at two or more different portions of a living being; an action potential measuring device capable of measuring an action potential of a heart of the living being; a heart sound measuring device capable of measuring a heart sound of the living being; and the computer according to claim 3.

5. A pulse wave analyzing method characterized by applying a rheological analysis to pulse wave data measured at two or more different portions of a human being and thereby calculating at least one parameter selected from an effective circulating volume (ECV, Ve) and a Young's modulus (E),
wherein the effective circulating volume (ECV, Ve) is given by a following formula A:
Ve=ρC²m·ΔVstₘₑₐₙ/mPm Formula A
(where ρ is a density; Cm is a mean pulse wave velocity; ΔVstₘₑₐₙ is a stroke volume; and mPm is a main mean blood pressure),
and wherein the Young's modulus (E) is given by a following formula B or following formulas C:
E_{A}=2ρRₒ·C²m/h_{A} Formula B
(where E_{A} is a Young's modulus of an aorta; Rₒ is an outer radius of the artery; and h_{A} is a wall thickness of the artery),
Eᵣᵤ=2ρrₒᵣᵤC²ᵣᵤ/hᵣᵤ
Eₗᵤ=2ρrₒₗᵤC²ₗᵤ/hₗᵤ
Eᵣₗ=2ρrₒᵣₗC²ᵣₗ/hᵣₗ
Eₗₗ=2ρrₒₗₗC²ₗₗ/hₗₗ Formula C
(where suffixes “ru”, “lu”, “rl”, and “ll” mean a right upper limb, a left upper limb, a right lower limb, and a left lower limb, respectively; rₒ is an outer radius of an artery of a limb; C is a pulse wave velocity; and h is a wall thickness of the artery).

6. A computer capable of implementing the pulse wave analyzing software according to claim 2.
